(19) United States
(12) Patent Application Publication
(10) Pub. No.: US 2026/0144428 A1
(43) Pub. Date: May 28, 2026

(54) Endoscopy Reading Assistance System

(71) Applicant: AIDOT INC., Seoul (KR)

(72) Inventor: Jae Hoon Jeong, Seongnam-si, Gyeonggi-do (KR)

(21) Appl. No.: 18/867,692

(22) PCT Filed: Jul. 25, 2022
(86) PCT No.: PCT/KR2022/010920

§ 371 Date: Aug. 8, 2025

(30) Foreign Application Priority Data
May 24, 2022 (KR) 10-2022-0063364

(51) Int. Cl. A61B 1/00; G06T 7/00; G16H 30/20; G16H 50/30

(52) U.S. Cl. CPC A61B 1/000094; A61B 1/000096; A61B 1/00045; G06T 7/0014; G16H 30/20; G16H 50/30; G06T 2200/24; G06T 2207/10068; G06T 2207/20084; G06T 2207/30096

(57) ABSTRACT

The present invention relates to an endoscopy reading assistance system configured to read a lesion in an endoscopy image and display a reading status and a reading result in real time, wherein the system includes a lesion reading unit which detects a lesion region in an endoscopy image input from endoscopy equipment by using a pre-trained deep neural network training model for lesion reading, and reads a risk level of a lesion in the detected lesion region, and a screen display control unit which displays an activation status of the lesion reading unit on a user interface screen of a computer system.

### TECHNICAL FIELD

The present invention relates to a system for reading lesions in endoscopy images, and particularly to an endoscopy reading assistance system that reads lesions in endoscopy images and displays a reading status and a reading result in real time.

### BACKGROUND ART

An endoscope may be used to diagnose conditions or detect lesions inside the body. A commonly used endoscopy method for acquiring images of the inside of the body is to insert a flexible tube attached with a camera into a digestive organ or the like through a patient's mouth or anus and capture images of the inside of the body.

Endoscopy is a test method that allows a more accurate diagnosis of a disease by observing mucous membranes with the naked eye through an endoscope to determine the presence or absence of lesions, and in some cases, by immediately performing a biopsy. For accurate endoscopy, it is necessary to accurately identify the location of lesions of benign and malignant diseases with the naked eye, but there is a significant deviation in detection rates of the location of lesions depending on the skill level of a practitioner. Since the deviation between detection rates in the morning and in the afternoon is significantly large, an endoscopic examination can be said to be a test method that requires a high level of concentration from a practitioner, and increases the fatigue of the practitioner.

Meanwhile, in recent years, reading assistance systems applied with various types of deep neural network models have been developed to increase the efficiency of endoscopy. Since such reading assistance systems are used in connection with endoscopy equipment, it is inconvenient for a practitioner in that the practitioner needs to pay attention not only to the operation of the endoscopy equipment but also to the operation of the reading assistance system.

### Prior Art Document

### Patent Document

(Patent Document 1) Korean Patent Laid-Open Publication No. 10-2020-0070062

(Patent Document 2) Korean Patent Laid-Open Publication No. 10-2020-0038121

### DISCLOSURE OF THE INVENTION

### Technical Problem

Accordingly, the present invention is an invention created to solve the above-described problem, and the main purpose of the present invention is to provide an endoscopy reading assistance system that allows the start and end of endoscopy to be performed without a separate operation of a reading assistance system to which a deep neural network is applied while a practitioner is performing the endoscopy.

Furthermore, another purpose of the present invention is to provide an endoscopy reading assistance system that assists a practitioner to intuitively recognize the driving status of a deep neural network and lesion detection results according to the driving of the deep neural network.

In addition, another object of the present invention is to provide an endoscopy reading assistance system that automatically detects the location of lesions by distinguishing between gastroscopy and colonoscopy, and displays risk levels of the detected lesions to the outside.

### TECHNICAL SOLUTION

An endoscopy reading assistance system according to an embodiment of the present invention to achieve the above-described purposes is a system installed and executable in a computer system connected with endoscopy equipment, and the system includes
a lesion reading unit configured to use a pre-trained deep neural network learning model for lesion reading, thereby detecting a lesion region in an endoscopy image input from the endoscopy equipment, and reading a risk level of a lesion in the detected lesion region, and
a screen display control unit configured to display an activation status of the lesion reading unit on a user interface screen of the computer system.

An endoscopy reading assistance system according to another embodiment of the present invention is also a system installed and executable in a computer system connected with endoscopy equipment, and the system includes
an internal body image determination unit configured to determine whether an endoscopy image input from the endoscopy equipment is an internal body endoscopy image, and
a screen display control unit configured to display, on a user interface screen of the computer system, an activation status of a lesion reading unit including a deep neural network learning model, if the endoscopy image is an internal body endoscopy image.

The above-described endoscopy reading assistance system may further include a lesion reading unit activated if the endoscopy image is an internal body endoscopy image, and configured to use a pre-trained deep neural network learning model for lesion reading, thereby detecting a lesion region in the internal body endoscopy image, and reading a risk level of a lesion in the detected lesion region, and
a freeze image acquisition unit configured to regard, as a lesion region, image frames in which the similarity between a predetermined number of adjacent frames among frames of the endoscopy image input from the endoscopy equipment exceeds a predetermined threshold, and transmit the image frames to the lesion reading unit.

Furthermore, the system may further include detected image storage unit configured to store an image of a frame unit detected or regarded as the lesion region, wherein the screen display control unit further counts and accumulates the number of detection of lesion regions and reading results by risk level, and displaying the number of detection and the reading results on the user interface screen.

In this case, the detected image storage unit may automatically change a filename every time the lesion reading unit, which is deactivated if the endoscopy image is an external body endoscopy image, is activated, thereby storing an image of the lesion region.

An endoscopy reading assistance system according to yet another embodiment of the present invention is a system installed and executable in a computer system connected with endoscopy equipment, and the system may include
an internal body image determination unit configured to determine whether an endoscopy image input from the endoscopy equipment is an internal body endoscopy image,
an examination site identification unit configured to identify an endoscopic examination site by using a deep neural network learning model pre-trained for the internal body endoscopy image,
a lesion reading unit by examination site selectively activated according to the result of the identified examination site, and configured to use a pre-trained deep neural network learning model for lesion reading, thereby detecting a lesion region in the internal body endoscopy image, and reading a risk level of a lesion in the detected lesion region, and
a screen display control unit configured to display an activation status of the activated lesion reading unit on a user interface screen of the computer system.

An endoscopy reading assistance system according to still another embodiment of the present invention is also a system which may include an internal body image determination unit configured to determine whether an endoscopy image input from the endoscopy equipment is an internal body endoscopy image,
a lesion reading unit configured to use a pre-trained deep neural network learning model for lesion reading, thereby detecting a lesion region in an endoscopy image input from the endoscopy equipment, and reading a risk level of a lesion in the detected lesion region, and
a detected image storage unit configured to automatically change a filename if the endoscopy image is an internal body endoscopy image, thereby storing an image of the detected lesion region.

In this case, the lesion reading unit is activated if the endoscopy image is an internal body endoscopy image, and deactivated if the same is an external body endoscopy image.

### Advantageous Effects

According to the above-described technical problem solving means, an endoscopy reading assistance system according to an embodiment of the present invention displays a driving status of a deep neural network learning model on a user interface screen, and thus, allows a specialist or others to intuitively recognize that a deep neural network is driving normally through the user interface screen without separately operating the system.

In addition, according to the present invention, if a lesion region is detected in an endoscopy image, an image of the lesion region is automatically classified by patient and stored in an album, so that a practitioner or others may be provided with convenience in which an endoscopy image by patient is automatically distinguished and stored without having to separately operate the system.

In addition, the present invention automatically diagnoses a risk level of a lesion by using a pre-trained deep neural network learning model and displays the result of the diagnosis in a status bar or the like, and thus, has an advantage providing objective and highly reliable diagnosis results regardless of the experience, ability, and skill level of a specialist, and
in addition, the present invention has an advantage of allowing lesion reading for a plurality of organs (stomach and large intestine) by using one system, and since a practitioner or others do not need to operate the system according to a different organ to be examined, the present invention has an effect of providing convenience in using the system.

### BRIEF DESCRIPTION OF THE DRAWINGS

FIG. 1 is an exemplary peripheral configuration diagram of an endoscopy reading assistance system according to an embodiment of the present invention.

FIG. 2 is another exemplary configuration diagram of an endoscopy reading assistance system according to an embodiment of the present invention.

FIG. 3 is an exemplary configuration diagram of an endoscopy reading assistance system according to yet another embodiment of the present invention.

FIG. 4 is an exemplary operation flow diagram for explaining the operation of an endoscopy reading assistance system according to an embodiment of the present invention.

FIG. 5 is an exemplary detailed flow diagram of a lesion detection and reading routine for an endoscopy image in FIG. 4.

FIG. 6 s an exemplary endoscopy image display diagram before, during, and at the end of endoscopy.

FIG. 7 is an exemplary diagram of a user interface screen according to an embodiment of the present invention.

FIG. 8 is an exemplary display diagram of a lesion region detection status according to an embodiment of the present invention.

FIG. 9 is a diagram for explaining a freeze image acquisition process.

FIG. 10 is an exemplary configuration diagram of an endoscopy reading assistance system according to still another embodiment of the present invention.

### MODE FOR CARRYING OUT THE INVENTION

A detailed description of the present invention described below refers to the accompanying drawings, which illustrate, by way of example, specific embodiments in which the present invention may be implemented to clarify the objectives, technical solutions, and advantages of the present invention. These embodiments are described in detail enough for those skilled in the art to be able to implement the present invention.

In addition, it will be understood by those skilled in the art that throughout the detailed description and claims of the present invention, ‘learning’ is a term that refers to performing machine learning according to a procedure, and is not intended to refer to a mental operation such as a human educational activity. In addition, throughout the detailed description and claims of the present invention, the word ‘include’ and variations thereof are not intended to exclude other technical features, additions, components, or steps. Other objects, advantages, and features of the present invention will be apparent to those skilled in the art from this description, and partly from the implementation of the present invention. The following examples and drawings are provided as examples, and are not intended to limit the present invention. Moreover, the present invention encompasses all possible combinations of the embodiments set forth herein. It should be understood that the various embodiments of the present invention are different from each other, but need not be mutually exclusive. For example, configurations and properties described herein may be implemented in other embodiments without departing from the spirit and scope of the invention in relation to one embodiment. In addition, it should be understood that the location or placement of individual components within each disclosed embodiment can be changed without departing from the spirit and scope of the invention. Therefore, the following detailed description is not intended to be taken in a limiting sense, and the scope of the present invention, if appropriately described, is limited only by the appended claims, along with all ranges equivalent to those claimed. In the drawings, similar reference numerals refer to the same or similar functions across various aspects.

Unless otherwise indicated herein clearly contradicted in the context, an item referred to in the singular may be in the plural, unless the context clearly dictates otherwise. In addition, in describing the present invention, when it is determined that detailed descriptions of related known compositions or functions may obscure the gist of the present invention, the detailed descriptions thereof are omitted.

Hereinafter, in order to facilitate the implementation of the present invention by those skilled in the art, preferred embodiments of the present invention will be described in detail with reference to the accompanying drawings.

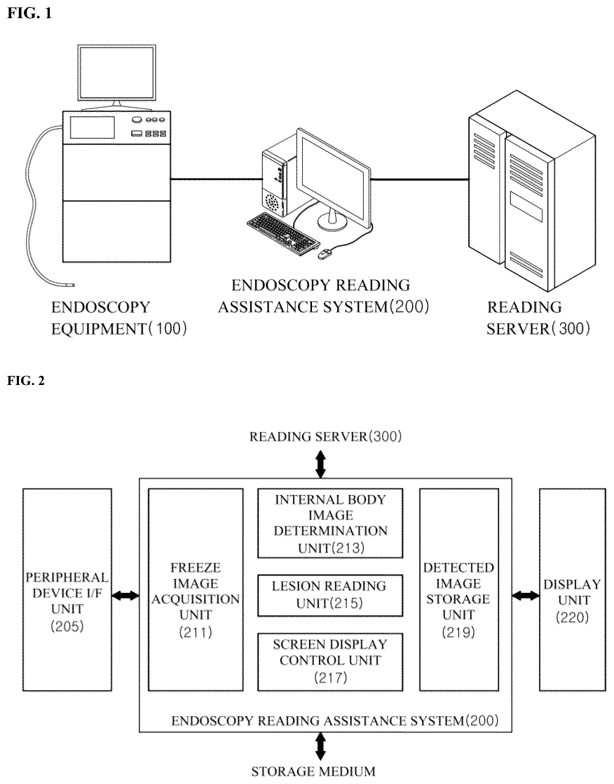

FIG. 1 is an exemplary peripheral the components diagram of an endoscopy reading assistance system according to an embodiment of the present invention.

An endoscopy reading assistance system 200 according to an embodiment of the present invention may be implemented as one independent system, and also, may be implemented as a collection (application program) of program data installed in a computer system of a professional (diagnostician, practitioner) and executable in a main processor of the computer system. In some cases, the system may also be implemented and executed in the form of an application program executable in a main processor (control unit) of endoscopy equipment.

FIG. 1 illustrates the endoscopy reading assistance system 200 installed in a computer system of a specialist, who is a practitioner, and depending on the implementation method thereof, the endoscopy reading assistance system 200 may automatically diagnose and display a risk level of a lesion in an image of a freeze state transmitted from endoscopy equipment 100, detect a lesion region in a real-time endoscopy image and automatically diagnose and display a lesion degree, that is, a risk level, of the detected lesion region, or detect a lesion region in a real-time endoscopy image and transmit the lesion region to a reading server 300, and receive and display the result of automatic diagnosis for the region from the reading server 300.

For reference, the endoscopy equipment 100 illustrated in FIG. 1 may be gastroscopy equipment, or colonoscopy equipment, and in some cases, may be endoscopy equipment that can perform both gastroscopy and colonoscopy. The endoscopy equipment 100 and the computer system in which the endoscopy reading assistance system 200 is installed are connected to each other through a cable and an image output terminal, so that the same endoscope image displayed on the endoscope equipment 100 may be displayed on a display unit 220 of the computer system of a specialist.

The endoscopy reading assistance system 200 according to an embodiment of the present invention illustrated in FIG. 1 may distinguish whether an endoscopy image is an internal body image or an external body image, and also, depending on the implementation method thereof, may distinguish whether the internal body image is a colonoscopy image or a gastroscopy image. In addition, the endoscopy reading assistance system 200 displays, to the outside, the current status, that is, an activation status or a deactivation status, of a deep neural network learning model which automatically detects a lesion region, and also, automatically determines whether an endoscopy image is an internal body image or an external body image and automatically creates a new filename to distinguish and store images by patient, thereby minimizing the need for a specialist to operate the system.

Hereinafter, in further describing the above-described endoscopy reading assistance system 200,

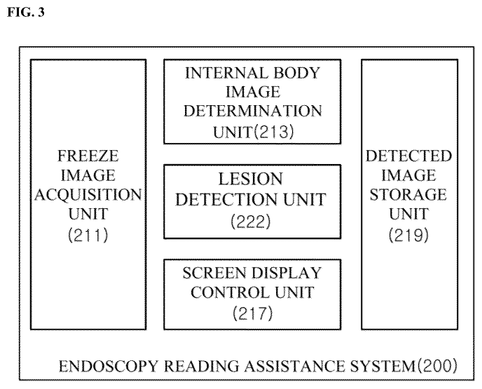

FIG. 2 illustrates a configuration diagram of an endoscopy reading assistance system 200 according to another embodiment of the present invention, and FIG. 3 illustrates a configuration diagram of an endoscopy reading assistance system 200 according to yet another embodiment of the present invention.

Before referring to FIG. 2, the endoscopy reading assistance system according to an embodiment of the present invention is basically a system installed and executable in a computer system connected to endoscopy equipment, and may include a lesion reading unit configured to use a pre-trained deep neural network learning model for lesion reading, thereby detecting a lesion region in an endoscopy image input from the endoscopy equipment, and reading a risk level of a lesion in the detected lesion region, and a screen display control unit configured to display an activation status of the lesion reading unit on a user interface screen of the computer system.

That is, the endoscopy reading assistance system 200 according to an embodiment of the present invention may be a system configured to display the status of a lesion reading unit on a user interface screen while the lesion reading unit is driving, in order to allow a practitioner to intuitively recognize a current operating status of the lesion reading unit while minimizing the need for the practitioner to operate the system unit.

In addition to the above-described basic configuration, the endoscopy reading assistance system 200 according to an embodiment of the present invention may include, as illustrated in FIG. 2,
an internal body image determination unit 213 configured to determine whether an endoscopy image input from the endoscopy equipment 100 is an internal body endoscopy image, and
a screen display control unit 217 configured to display, on a user interface screen of the computer system, an activation status of a lesion reading unit 215 including a deep neural network learning model, if the endoscopy image is an internal body endoscopy image.

The screen display control unit 217 may display an activation status or a deactivation status of the lesion reading unit 215 on an icon, a status bar, or the like, and also, may count and accumulate reading results by risk of a lesion region and the number of detection of lesion regions, and display a patient information button, an album button, a backup button, an environment setting button, a loading icon, and the like on the user interface screen.

The internal body image determination unit 213 may set, as a determination variable, one of the brightness of an endoscopy image, average brightness of the same, and degree of change in the same between frames, thereby determining whether an endoscopy image is of an internal body or an external body. Of course, the internal body image determination unit 213 may also determine whether an endoscopy image is an internal body image by including a deep neural network learning model for internal body determination in which images of an internal body have been pre-trained. That is, if a received endoscopy image has features of a pre-trained internal body image, the received endoscopy image may be determined as an internal body image. The pre-trained deep neural network learning model for internal body determination may be a learning model created by learning features of both a gastroscopy image and a colonoscopy image, or the features of only a gastroscopy image or a colonoscopy image.

The internal body image determining unit 213 activates the lesion reading unit 215 if an endoscopy image is an internal body endoscopy image, and deactivates the lesion reading unit 215 if the endoscopy image is an external body endoscopy image.

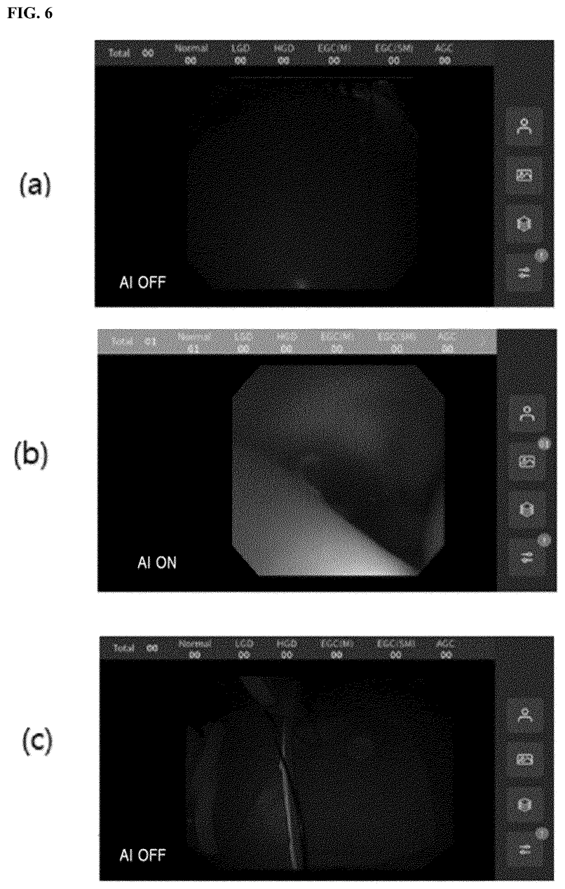

For reference, before endoscopy and at the end of the endoscopy, an external body image is generally obtained as an image darker than an internal body image as illustrated in (a) and (c) of FIG. 6, so that it is possible to determine whether an endoscopy image is an internal body image or an external body image by using the pre-trained deep neural network learning model as well as the brightness of the endoscopy image, average brightness of the same, and degree of change in the same between frames.

Meanwhile, the endoscopy reading assistance system 200 according to yet another embodiment of the present invention may further include, in addition to the internal body image determination unit 213 and the screen display control unit 217, a lesion reading unit 215 activated if an endoscopy image is an internal body endoscopy image, and configured to use a pre-trained deep neural network learning model for lesion reading, thereby detecting a lesion region in the internal body endoscopy image, and reading a risk level of a lesion in the detected lesion region.

The deep neural network learning model for lesion reading (Convolution Neural Network: CNN) may have a network structure in which a convolution layer and a pooling layer are repeated between an input layer and a fully connected layer, or may have a structure as published in Korean Application No. 10-2020-0007623, which was previously filed by the applicant of the present application, that is, a structure including a group of convolution layers and a group of deconvolution layers respectively processing a convolution operation and a deconvolution operation in parallel in a pooling layer and any one convolution layer among repeated convolution layers in order to mitigate noise, and including an add layer which combines feature maps that have passed through the group of convolution layers and the group of deconvolution layers into one map and transmits the one map to the fully connected layer.

The deep neural network learning model for lesion reading is a model built by pre-training endoscopy image data (training data) in which lesion regions and (or) risk levels of lesions are marked by a specialist through a deep learning algorithm, and detects a lesion region in a pre-processed endoscopy image in a diagnosis mode, and then diagnose a risk level of a lesion in the detected lesion region.

For reference, in the deep neural network learning model for lesion reading, a pair (x, y) of an input (x) and its corresponding one output (y) is learned. The input may be an endoscopy image, and the output may be, for example, a risk level of a lesion.

In addition, as each of the deep neural network learning models used in an embodiment of the present invention, a modified DenseNet-based convolutional neural network applied with hyper-parameter tuning may be used.

In some cases, the lesion reading unit 215 may be configured to include a deep neural network learning model for lesion region detection and a deep neural network learning model for lesion diagnosis.

The above-described lesion reading unit 215 is a deep neural network learning model which has pre-trained features of a gastroscopy image or a colonoscopy image, but may include both a deep neural network learning model which has pre-trained the features of an endoscopy image and a deep neural network learning model which has pre-trained the features of a colonoscopy image. In this case, an internal body endoscopy image is distinguished by whether it is a gastroscopy image or a colonoscopy image, and a deep neural network learning model corresponding thereto is activated.

For reference, if a deep neural network learning model for lesion reading is a diagnosis model for gastroscopy images, a risk level of a lesion may be diagnosed and displayed as Low Grade Dysplasia (LGD), High Grade Dysplasia (HGD), Early Gastric Cancer (EGC), and Advanced Gastric Cancer (AGC), and if it is a diagnosis model for colonoscopy images, the model may be pre-trained to be able to diagnose a cancer, thereby diagnosing a lesion, and display a risk level corresponding thereto.

As an additional embodiment that may be modified, the endoscopy reading assistance system 200 may further include, in addition to the internal body image determination unit 213, the lesion reading unit 215, and the screen display control unit 217 described above, a freeze image acquisition unit 211 configured to regard, as a lesion region (i.e., regard as a freeze image by a specialist), image frames in which the similarity between a predetermined number of adjacent frames among frames of the endoscopy image input from the endoscopy equipment 100 exceeds a predetermined threshold, and transmit the image frames to the lesion reading unit 215.

As another additional embodiment that may be modified, the endoscopy reading assistance system 200 may further include, in addition to the above-described components, a detected image storage unit 219 configured to store, in a storage medium, an image (a freeze image) of a frame unit detected as a lesion region or automatically regarded as a lesion region. In this case, the screen display control unit 217 counts and accumulates the number of detection of lesion regions and reading results by risk level, and displays the number of detection and the reading results on the user interface screen.

In addition, the detected image storage unit 219 automatically changes a filename every time the lesion reading unit 215, which is deactivated if the endoscopy image is an external body endoscopy image, is newly activated, thereby storing a newly detected lesion region image. This is to assume that it is a new patient's endoscopy if an endoscopy image is regarded as an internal body image again after a patient's endoscopy is completed and to distinguish and store a lesion region by patient without a separate operation by a specialist.

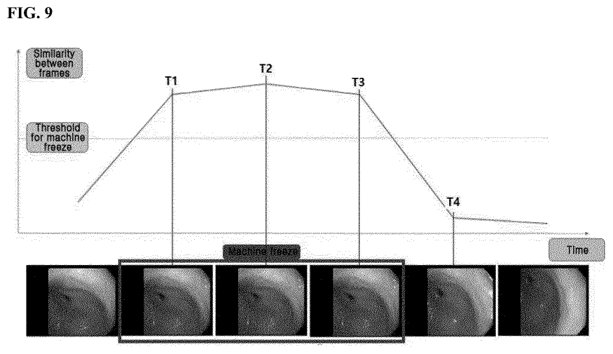

The freeze image acquisition unit 211 acquires, as a freeze image (understood as capture), image frames (image frames in T1, T2, and T3) in which the similarity between frames among frames of an endoscopy image exceeds a predetermined threshold as illustrated in FIG. 9. Here, it is preferable to understand the image frames in which the similarity between frames among frames of an endoscopy image exceeds a predetermined threshold as not only a state in which a specialist regards the image frames as a lesion region and machine-freezes the same, but also a state in which the same image frames are obtained since an moving endoscopy camera stops moving.

When machine-freezed by a specialist on the endoscopy equipment 100 side, a freezed endoscopy image is paused and displayed, so that it can be said that the similarity between image frames at this time is very high. As described above, if the similarity between frames exceeds a predetermined threshold, it is preferable for the endoscopy reading assistance system 200 to recognize that an endoscopy image is freezed on the endoscopy equipment 100 side and automatically diagnose whether there is a lesion in the image. Therefore, an auto-freezed image is first regarded as a lesion region, stored, and processed to be subjected to lesion diagnosis.

Meanwhile, a peripheral device I/F 205 not described in FIG. 2 illustrates an interface unit configured to interface an endoscope image between the endoscopy equipment 100 and the computer system, and a display unit 220 illustrates a display unit constituting the computer system. Since it is obvious to those skilled in the art that the computer system in which the endoscopy reading assistance system 200 according to an embodiment of the present invention is installed is not only equipped with a storage medium for storing information, but also a reading server 300 located externally and a communication unit (not shown) for transmitting and receiving the information, SO that a detailed description thereof will be omitted. In addition, since it is also known in the field of medical imaging devices for diagnosing a lesion by using a deep neural network that the endoscopy reading assistance system 200 including the lesion reading unit 215 further includes a pre-treatment unit configured to remove blood, texts, biopsy instruments, and the like from an endoscopy image, so that a detailed description thereof will be omitted.

FIG. 3 illustrates a configuration diagram of the endoscopy reading assistance system 200 according to yet another embodiment of the present invention, which is identical to the configuration illustrated in FIG. 2 except for a lesion detection unit 222. Accordingly, the lesion detection unit 222 and the screen display control unit 217 are further described in the configuration illustrated in FIG. 3.

The lesion detection unit 222 illustrated in FIG. 3 is activated by the internal body image determination unit 213 if an endoscopy image is an internal body endoscopy image, and detects a lesion region in the internal body endoscopy image by using a pre-trained deep neural network learning model for lesion detection, and transmits the detected lesion region to the reading server 300.

The screen display control unit 217 illustrated in FIG. 3 counts and accumulates reading results by risk of the lesion region transmitted from the reading server 300 and the number of detection of lesion regions, and displays the reading results and the number of detection on a user interface screen displayed on the display unit 220.

That is, the endoscopy reading assistance system 200 illustrated in FIG. 3 automatically detects only a lesion region in a gastroscopy or colonoscopy image and transmits the lesion region to the reading server 300 to automatically diagnose a lesion, and displays the automatic diagnosis result and a current operation status of the lesion detection unit 222 to the outside.

Each endoscopy reading assistance system 200 described above by embodiment may further include a technical component, e.g., a detection alarm unit, for notifying a specialist by alarm when a lesion region is detected.

Hereinafter, the operation of the endoscopy reading assistance system 200 described above will be described in more detail, and an operation of diagnosing a gastroscopy image will be described below.

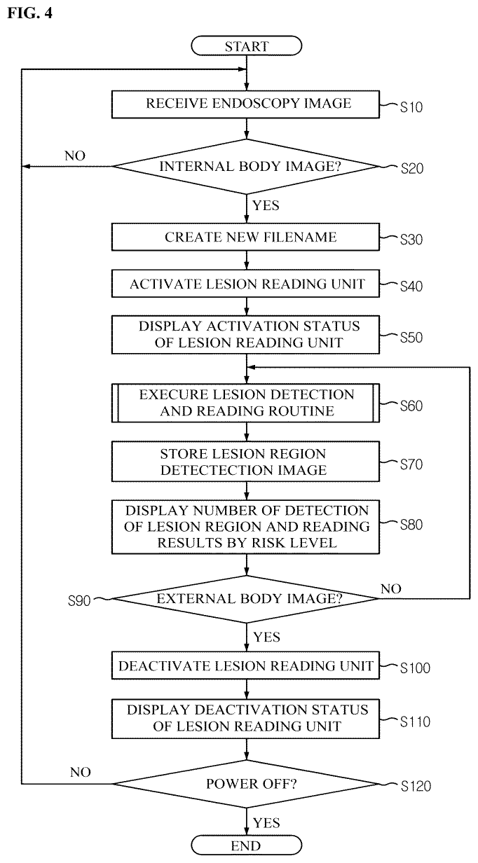

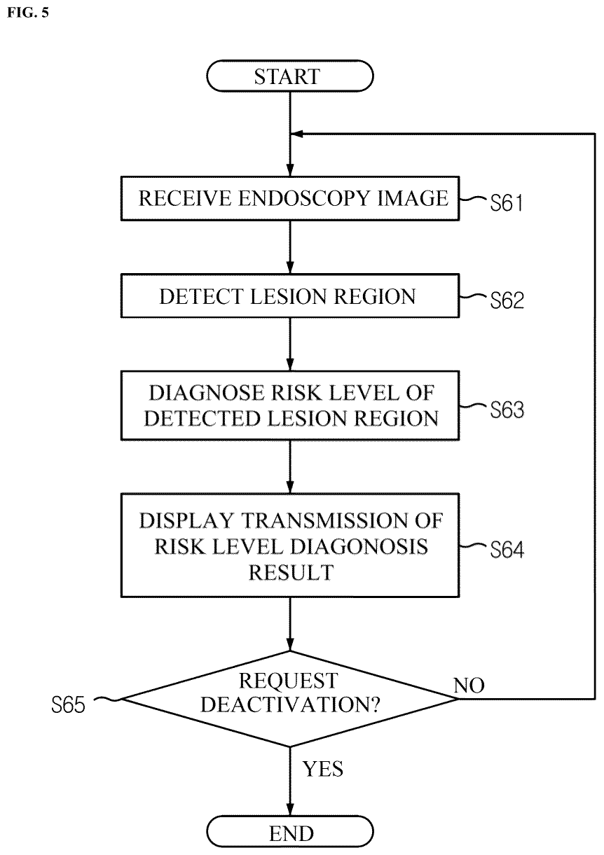

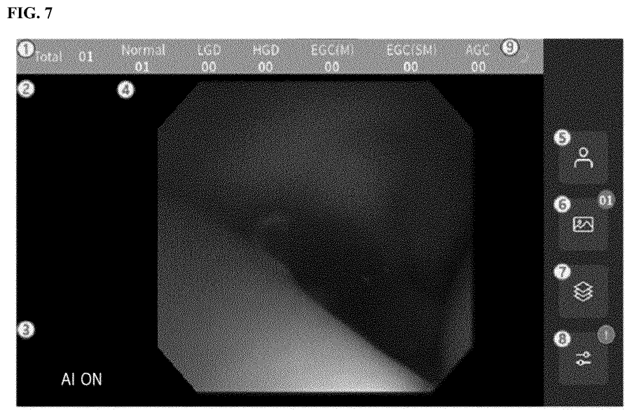

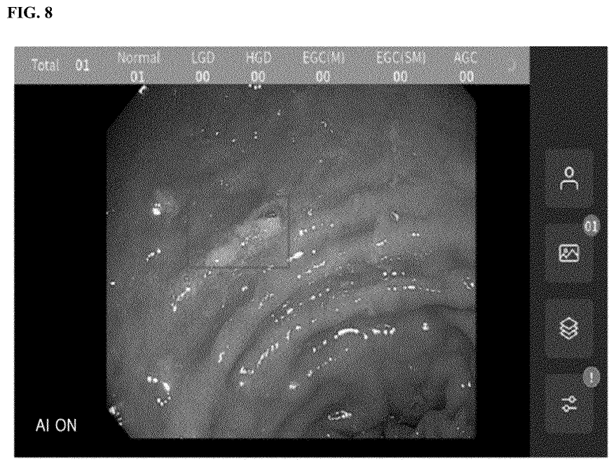

FIG. 4 illustrates an operation flow diagram for explaining the operation of the endoscopy reading assistance system according to an embodiment the present invention, and FIG. 5 illustrates a detailed flow diagram of a lesion detection and reading routine for an endoscopy image in FIG. 4. FIG. 6 displays an endoscopy image before, during, and at the end of endoscopy, FIG. 7 illustrates a user interface screen according to an embodiment of the present invention, and FIG. 8 illustrates a display screen a lesion region detection status according to an embodiment of the present invention.

Prior to automatic reading of a lesion in an endoscopy image, the endoscopy reading assistance system 200 needs to learn a deep neural network learning model for lesion reading through a learning mode.

For example, a specialist marks a lesion region and input information on a risk level (degree) of a lesion for a gastroscopy image. A large number of endoscopy image frames in which lesion regions and information on risk levels of regions are marked or input are transmitted to a deep neural network learning model for lesion reading which has a deep neural network structure according to the command of a specialist.

Accordingly, the deep neural network learning model for lesion reading learns learning data, that is, features of an image in which a lesion region is marked in a gastroscopy image, and completes the learning of a model for predicting any one of normal/LGD/HGD/EGC/AGC as a risk level of a lesion for the gastroscopy image through tests and verification steps.

When the learning of the deep neural network learning model for lesion reading is completed, it is possible to detect a lesion region in a gastroscopy image and automatically detect a risk level on the basis of the learning model.

In addition, in order to automatically determine whether an endoscopy image is an internal body image or an external body image, a specialist sets learning data to allow features of images, which are at an initial position at which a stomach begins or at a position just before the initial position in an gastroscopy image, to be learned, thereby allowing learning of a deep neural network learning model for internal body determination, and then it is possible to automatically read whether an endoscopy image is an internal body image on the basis of the deep neural network learning model for internal body determination.

As described above, if the learning data is set to allow learning of a deep neural network learning model to learn features of a lesion site, features of an internal body image, features of a gastroscopy image, and features of an internal organ endoscopy image, a gastroscopy image and a colonoscopy image are automatically distinguished based on the learning, thereby selectively activating a deep neural network learning model for gastroscopy lesion reading or colonoscopy lesion reading to automatically read a lesion of a stomach or a colon.

Hereinafter, referring to FIG. 4, a gastroscopy image obtained through an endoscope is first displayed on a display unit of the endoscopy equipment 100, and is also received (Step S10) on a specialist's PC in which the endoscopy reading assistance system 200 is installed, and displayed on the display unit 220.

If endoscopy is not being performed, an endoscope camera is in a state of being left to stand at a specific position, so that images with no change will be received continuously, or images with no shape of a specific subject will be received as shown in (a) of FIG. 6.

Then, the internal body image determination unit 213 of the endoscopy reading assistance system 200 according to an embodiment of the present invention determines whether an endoscopy image received or input is an internal body image (Step S20). As a determination method, the deep neural network learning model which has learned the features of an internal body image may be used, and the brightness of an endoscopy image, average brightness of the same, and degree of change in the image may be used.

If the endoscopy image is determined to be an internal body image, it is regarded that endoscopy of a patient has begun, which is transmitted to the detected image storage unit 219 to induce a new filename to be created (Step S20) such that images of lesion regions to be detected later are distinguished and stored by patient. The internal body image determination unit 213 may also directly create a new filename. The new filename created may be present in an album folder.

In addition, the internal body image determination unit 213 activates the lesion reading unit 215 (Step S40) and transmits the activation to the screen display control unit 217, so that the screen display control unit 217 displays an activation status (A.I ON) of the lesion reading unit 215 including a deep neural network learning model on the user interface screen (Step S50) as shown in (b) and FIG. 7.

Following the activation, the lesion reading unit 215 enters a lesion automatic reading state for an endoscopy image. That is, the lesion reading unit 215 executes a lesion detection and reading routine (Step S60). The lesion detection and reading routine is further described with reference to FIG. 5.

The lesion reading unit 215 receives an endoscopy image (Step S61 step) and pre-processes the received image. The pre-processing for removing unnecessary regions and objects in order to read a lesion may be designed differently depending on the type of an image to be read (gastroscopy, colonoscopy), and typically, pre-processing may be performed such that texts, auxiliary diagnostic instruments, blood, and images of organs other than a target of observation may be removed if necessary. The above-described image pre-processing process is performed by a pre-processing unit located inside the lesion reading unit 215.

On the other hand, the lesion reading unit 215 uses a pre-trained deep neural network learning model for lesion reading to detect a lesion region in real time in the pre-processes endoscopy image frame (Step S62). If a lesion region is detected, the lesion reading unit 215 transmits coordinate information for displaying the lesion region to the screen display control unit 217. Accordingly, the screen display control unit 217 displays and outputs an endoscopy image frame marked with the lesion region (square box) as illustrated in FIG. 8, and may additionally display the status of lesion region detection by sound alarm.

In addition, the lesion reading unit 215 captures a lesion region upon detection and transmits the detected lesion region to the detected image storage unit 219 to request for storage. The captured image to be transmitted is transmitted together with the time of creation of the corresponding image as additional information, and transmitted together with the result of risk level reading for the corresponding lesion region to be transmitted later. Accordingly, a newly created filename (usually, a patient's name) is stored along with lesion regions sequentially detected together with the result of reading (diagnosis) for the corresponding lesion region (Step S70).

The lesion reading unit 215 uses a pre-trained deep neural network learning model for lesion reading to automatically read a risk level of a lesion in the detected lesion region (Step S63). Subsequently, the lesion reading unit 215 transmits the result of diagnosis (reading) of the risk level of the detected lesion region in real time to the detected image storage unit 219 and the screen display control unit 217 (Step S64).

Accordingly, the screen display control unit 217 counts and accumulates the number of detection of lesion regions and the reading results by risk which are transmitted in real time and displays the number of detection and the reading results on the user interface screen as shown in FIG. 7 (Step S80).

Referring to FIG. 7, a status bar \textcircled{1} may be located at the top of the user interface screen. The status bar \textcircled{1} displays the total number of images automatically detected as lesion regions and image frames automatically freezed, and displays the results of risk level diagnosis (Normal, LGD, HGD, EGC, AGC) for each detected image frame. In addition, an icon (A.I. ON) indicating the activation status of the lesion reading unit 215 including a deep neural network learning model is displayed together, and a number of buttons \textcircled{5}, \textcircled{6}, \textcircled{7}, and \textcircled{8} are displayed on the left side of the screen.

The button \textcircled{5} is a patient information button used to move to a patient information screen, and the button \textcircled{6} is an album button used to confirm that freezes and detected image frames are stored by patient. The number displayed on the album button displays the number of image frames stored. The button \textcircled{7} is a backup button, and is used to move to a backup screen when clicked. The button \textcircled{8} is a setting button, and is used to move to a setting screen when clicked. Through the settings screen, a specialist or others may set system variables. The button \textcircled{9} is a loading icon and displayed when AI reading is being performed.

On the other hand, when a specialist or others pause and freeze an endoscopy camera while the lesion reading unit 215 is automatically detecting a lesion region, the freeze image acquisition unit 211 automatically acquires the freeze image as an observation image and stores the image in the detected image storage unit 219, and transmits the same to the lesion reading unit 215 to allow the reading of the freeze image.

For reference, the frieze image acquisition unit 211 may acquire, as freeze images, image frames in which the similarity between frames among frames of an endoscopy image exceeds a predetermined threshold as illustrated in FIG. 9, e.g., image frames at points of time of T1, T2, and T3 illustrated in FIG. 9.

For these freeze images, the lesion reading unit 215 reads risk levels of lesions by using a pre-trained deep neural network learning model for lesion reading (Step S63). The risk levels read and the number of freeze images are also accumulated with lesion region detection images and are displayed together on the user interface screen.

As described above, the lesion reading unit 215 detects a lesion region and automatically reads a risk level for a received endoscopy image until there is a deactivation request (Step S65). The deactivation request is transmitted by the internal body image determination unit 213. That is, if an endoscopy image (external body image) such as (c) of FIG. 6 is received (Step S90 of FIG. 4) while endoscopy is being performed after the determination of an internal body image, the internal body image determination unit 213 determines that the endoscopy for a patient has been completed, and transmits the status to the lesion reading unit 215 and the screen display control unit 217. Accordingly, the lesion reading unit 215 transitions to a deactivation state (Step S100), and the screen display control unit 217 displays the deactivation status (A.I. OFF) of the lesion reading unit 215 on the user interface screen (Step S110). For reference, (a) of FIG. 6 illustrates the screen before the examination, (b) thereof illustrates the screen during the examination, and (c) thereof illustrates the screen at the end of the examination.

While the lesion reading unit 215 is in the deactivation state, if the internal body image determination unit 213 determines an internal body image again, internal image again, it means that endoscopy is performed on a new patient, so that a new filename is created, and the lesion reading unit 215 is activated to allow detection of a lesion region and reading of a lesion, and the activation status of the lesion reading unit 215 is again displayed on the user interface screen.

As a result, a specialist or others may intuitively recognize that the lesion reading unit 215 is driving normally through the user interface screen without separately operating the system, and may be provided with convenience of not having to enter filenames, and the like required to distinguish and store endoscopy images for each patient.

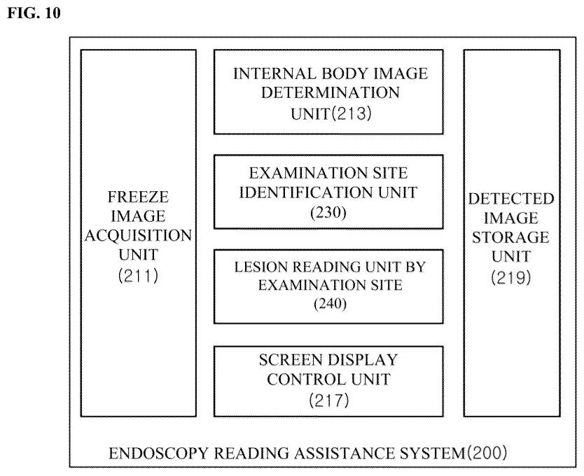

In the above embodiment, it has been described that an endoscopy image is primarily determined whether it is an internal body image, and then if determined to be an internal body image, a lesion region is detected in the endoscopy image to automatically read a risk level of a lesion in the corresponding lesion region, but as illustrated in FIG. 10, it is possible to build an endoscopy reading assistance system 200 including
an internal body image determination unit 213 configured to determine whether an endoscopy image input from endoscopy equipment 100 is an internal body endoscopy image,
an examination site identification unit 230 configured to identify an endoscopy site, i.e., a stomach and a colon, by using a deep neural network learning model pre-trained for the internal body endoscopy image,
a lesion reading unit 240 by examination site selectively activated according to the result of the identified examination site, and configured to use a pre-trained deep neural network learning model for lesion reading, thereby detecting a lesion region in the internal body endoscopy image, and reading a risk level of a lesion in the detected lesion region, and
a screen display control unit 217 configured to display an activation status of the activated lesion reading unit 240 on a user interface screen of the computer system. The lesion reading unit 240 by examination site includes a gastroscopy lesion reading unit configured to read a lesion in a gastroscopy image, and a colonoscopy lesion reading unit configured to read a lesion in a colonoscopy image.

The above-described endoscopy reading assistance system 200 may also further include the freeze image acquisition unit 211 and the detected image storage unit 219 described with reference to FIG. 2.

An endoscopy reading assistance system 200 illustrated in FIG. 10 has a structure in which an endoscopy image is primarily determined whether it is an internal body image, and then another deep neural network learning model is used to automatically read an examination site, thereby activating a deep neural network learning model optimized for the corresponding examination site to automatically read a lesion, and thus, has an advantage of allowing lesion reading for a plurality of organs by using one system, and since a practitioner or others do not need to operate the system according to a different organ to be examined, the present invention has an effect of providing convenience in using the system.

In the above embodiment, it has been described that the endoscopy reading assistance system 200 detects a lesion region in an endoscopy image and automatically diagnoses or reads a lesion in the lesion region, but as illustrated in FIG. 3, a system may be configured such that the detection of a lesion region is performed in the endoscopy reading assistance system 200, and the reading of a lesion is performed in a reading server 300. In the above-described system, a lesion detection unit 222 is activated if an endoscopy image is an internal body endoscopy image, and detects a lesion region in the internal body endoscopy image by using a pre-trained deep neural network learning model for lesion detection, and transmits the detected lesion region to the reading server 300. A screen display control unit 217 counts and accumulates reading results by risk of the lesion region transmitted from the reading server 300 and the number of detection of lesion regions, and displays the reading results and the number of detection on a user interface screen, and also displays an activation status of the lesion detection unit 222.

The endoscopy reading assistance system 200 according to still another embodiment of the present invention that may be modified may be configured to only include an internal body image determination unit 213 configured to determine whether an endoscopy image input from endoscopy equipment 100 is an internal body endoscopy image,
a lesion reading unit 215 configured to use a pre-trained deep neural network learning model for lesion reading, thereby detecting a lesion region in an endoscopy image input from the endoscopy equipment, and reading a risk level of a lesion in the detected lesion region, and
a detected image storage unit 215 configured to automatically change a filename if the endoscopy image is an internal body endoscopy image, thereby storing an image of the detected lesion region. Even in the above-described a system, the lesion reading unit 215 is activated if an endoscopy image is an internal body endoscopy image, and is deactivated if the same is an external body endoscopy image, and the status of the unit may be displayed on a user interface screen by a screen display control unit 217.

In the endoscopy reading assistance systems 200 according to various embodiments of the present invention described above, if a lesion region is detected in an endoscopy image, an image of the lesion region is automatically classified by user and stored in an album, SO that a practitioner such as a specialist may be provided with convenience of being able to further diagnose the degree of a lesion by intensively observing an image frame with a mark of a legion region through searching an album later.

In addition, the system of the present invention automatically diagnoses a risk level of a lesion by using a pre-trained deep neural network learning model and displays the result of the diagnosis in a status bar or the like, and thus, has an advantage of providing objective and highly reliable diagnosis results regardless of the experience, ability, and skill level of a specialist.

On the other hand, in the above embodiment, a system has been described wherein the endoscopy reading assistance system 200 is installed in a specialist's PC to detect a lesion in an endoscopy image or read a risk level of the lesion, but the system may also be implemented as an embedded system wherein the above-described endoscopy reading assistance system 200 is installed in endoscopy equipment 100 or is executed in a main processor of the endoscopy equipment 100.

Based on the descriptions of the above embodiments, those skilled in the art can clearly understand that the present invention may be implemented through a combination of software and hardware or may be implemented with hardware alone. Objects of the technical solution of the present invention or parts contributing to the prior art may be implemented in the form of program instructions that can be executed by various computer components and recorded on a machine-readable recording medium. The machine-readable recording medium may include, alone or in combination, program instructions, data files, data structures, and the like.

Although the present invention has been described above with specific details such as specific components and limited embodiments and drawings, these are provided only to facilitate a more general understanding of the present invention, and the present invention is not limited to the above embodiments, and those skilled in the art to which the present invention belongs can make various modifications and variations from these descriptions. Therefore, it should be understood that the spirit of the present invention is not limited to the above-described embodiments, and not only the scope of the patent claims described below, but also all things that are equal to or equivalently modified from the claims are said to fall within the scope of the spirit of the present invention.

1. endoscopy reading assistance system installed and executable in a computer system connected with endoscopy equipment, and comprising:
a lesion reading unit configured to use a pre-trained deep neural network learning model for lesion reading, thereby detecting a lesion region in an endoscopy image input from the endoscopy equipment, and reading a risk level of a lesion in the detected lesion region; and
a screen display control unit configured to display an activation status of the lesion reading unit on a user interface screen of the computer system.

2. An endoscopy reading assistance system installed and executable in a computer system connected with endoscopy equipment, and comprising:
an internal body image determination unit configured to determine whether an endoscopy image input from the endoscopy equipment is an internal body endoscopy image; and
a screen display control unit configured to display, on a user interface screen of the computer system, an activation status of a lesion reading unit including a deep neural network learning model, if the endoscopy image is an internal body endoscopy image.

3. The endoscopy reading assistance system of claim 2, further comprising a lesion reading unit activated if the endoscopy image is an internal body endoscopy image, and configured to use a pre-trained pre-trained deep neural network learning model for lesion reading, thereby detecting a lesion region in the internal body endoscopy image, and reading a risk level of a lesion in the detected lesion region.

4. The endoscopy reading assistance system of claim 3, further comprising a freeze image acquisition unit configured to regard, as a lesion region, image frames in which the similarity between a predetermined number of adjacent frames among frames of the endoscopy image input from the endoscopy equipment exceeds a predetermined threshold, and transmit the image frames to the lesion reading unit.

5. The endoscopy reading assistance system of claim 3 or claim 4, further comprising a detected image storage unit configured to store, in a storage medium, an image of a frame unit detected or regarded as the lesion region, wherein the screen display control unit counts and accumulates the number of detection of lesion regions and reading results by risk level, and displays the number of detection and the reading results on the user interface screen.

6. The endoscopy reading assistance system of claim 4, wherein the detected image storage unit automatically changes filename every time the lesion reading unit, which is deactivated if the endoscopy image is an external body endoscopy image, is activated, thereby storing an image of the lesion region.

7. The endoscopy reading assistance system of claim 2, further comprising a lesion detection unit activated if the endoscopy image is an internal body endoscopy image, and configured to use a pre-trained deep neural network learning model for lesion reading, thereby detecting a lesion region in the internal body endoscopy image, and transmitting the detected lesion region to a reading server, wherein the screen display control unit counts and accumulates reading results by risk of the detected lesion region transmitted from the reading server and the number of detection of lesion regions, and displays the reading results and the number of detection on the user interface screen.

8. The endoscopy reading assistance system of claim 7, further comprising a freeze image acquisition unit configured to regard, as a lesion region, image frames in which the similarity between a predetermined number of adjacent frames among frames of the endoscopy image input from the endoscopy equipment exceeds a predetermined threshold, and transmit the image frames to the lesion detection unit.

9. The endoscopy reading assistance system of claim 7 or claim 8, further comprising a detected image storage unit configured to store a lesion region image of a frame unit to be transmitted to the reading server.

10. The endoscopy reading assistance system of claim 9, wherein the detected image storage unit automatically changes a filename every time the lesion detection unit, which is deactivated if the endoscopy image is an endoscopy image of an external body, is activated, thereby storing the lesion region image.

11. The endoscopy reading assistance system of any one of claim 2, claim 3, claim 4, claim 7, and claim 8, wherein the internal body image determination unit sets, as a determination variable, one of the brightness of an endoscopy image, average brightness of the same, and degree of change in the same between frames, thereby determining whether an endoscopy image is of an internal body or an external body.

12. The endoscopy reading assistance system of any one of claim 2, claim 3, claim 4, claim 7, and claim 8, wherein the internal body image determination unit detects features of an internal body image from the endoscopy image input from the endoscopy equipment by using a pre-trained deep neural network learning model for internal body determination, thereby determining whether the endoscopy image is an internal body image.

13. The endoscopy reading assistance system of claim 12, wherein the pre-trained deep neural network learning model for internal body determination is a deep neural network learning model created by pre-learning features of one of a gastroscopy image or a colonoscopy image.

14. An endoscopy reading assistance system installed and executable in a computer system connected with endoscopy equipment, and comprising:
an internal body image determination unit configured to determine whether an endoscopy image input from the endoscopy equipment is an internal body endoscopy image;
an examination site identification unit configured to identify an endoscopy site by using a deep neural network learning model pre-trained for the internal body endoscopy image;
a lesion reading unit by examination site selectively activated according to the result of the identified examination site, and configured to use a pre-trained deep neural network learning model for lesion reading, thereby detecting a lesion region in the internal body endoscopy image, and reading a risk level of a lesion in the detected lesion region; and
a screen display control unit configured to display an activation status of the activated lesion reading unit on a user interface screen of the computer system.

15. The endoscopy reading assistance system of claim 14, further comprising a freeze image acquisition unit configured to regard, as a lesion region, image frames in which the similarity between a predetermined number of adjacent frames among frames of the endoscopy image input from the endoscopy equipment exceeds a predetermined threshold, and transmit the image frames to the lesion reading unit.

16. The endoscopy reading assistance system of claim 14 or claim 15, further comprising a detected image storage unit configured to store an image of a frame unit detected or regarded as the lesion region, wherein the screen display control unit counts and accumulates the number of detection of lesion regions and reading results by risk level, and displays the number of detection and the reading results on the user interface screen.

17. The endoscopy reading assistance system of claim 16, wherein the detected image storage unit automatically changes a filename every time the lesion reading unit, which is deactivated if the endoscopy image is an external body endoscopy image, is activated, thereby storing an image of the lesion region.

18. The endoscopy reading assistance system of claim 14 or claim 15, wherein the lesion reading unit by examination site includes:
a gastroscopy lesion reading unit configured to read a lesion in a gastroscopy image; and
a colonoscopy lesion reading unit configured to read a lesion in a colonoscopy image.

19. An endoscopy reading assistance system installed and executable in a computer system connected with endoscopy equipment, and comprising:
an internal body image determination unit configured to determine whether an endoscopy image input from the endoscopy equipment is an internal body endoscopy image;
a lesion reading unit configured to use a pre-trained deep neural network learning model for lesion reading, thereby detecting a lesion region in an endoscopy image input from the endoscopy equipment, and reading a risk level of a lesion in the detected lesion region; and
a detected image storage unit configured to automatically change a filename if the endoscopy image is an internal body endoscopy image, thereby storing an image of the detected lesion region.

20. The endoscopy reading assistance system of claim 19, wherein the lesion reading unit is activated if the endoscopy image is an internal body endoscopy image, and deactivated if the same is an external body endoscopy image.
